(19) United States
(12) Patent Application Publication
(10) Pub. No.: US 2007/0037161 A1
(43) Pub. Date: Feb. 15, 2007

(54) Motoneuronotrophic factor gene sequences

(76) Inventor: Bob Baoguo Xue, Union City, CA (US)

Correspondence Address: DUANE MORRIS LLP, 101 WEST BROADWAY, SUITE 900, SAN DIEGO, CA, 92101-8285, US

(21) Appl. No.: 10/578,158

(22) PCT Filed: Nov. 8, 2004
(86) PCT No.: PCT/US04/38651

§ 371 Date: May 4, 2006

Related U.S. Application Data
(60) Provisional application No. 60/518,581, filed on Nov. 7, 2003.

(51) Int. Cl. C12Q 1/68; C07H 21/04; C12P 21/06; C07K 14/475; C07K 14/52

(52) U.S. Cl. 435/6; 536/23.5; 435/69.1; 435/320.1; 435/325; 530/399

(57) ABSTRACT

The invention is directed nucleic acids associated with sequences encoding motoneuronotrophic factors, which promote the survival, growth, proliferation, or maintenance of mammalian neurons, polypeptides encoded by the sequences, and assays for detecting the sequences.

### RELATED APPLICATIONS

The present application claims the benefit of U.S. provisional application No. 60/518,581 filed Nov. 7, 2003, which is incorporated herein by reference in its entirety.

### FIELD OF INVENTION

The present invention relates to human genes encoding a specialized group of proteins, which promote the growth, maintenance, survival, and functional capabilities of selected populations of neurons.

### BACKGROUND OF INVENTION

Neuronotrophic factors (NTFs) are a specialized group of proteins which function to promote the survival, growth, maintenance, and functional capabilities of selected populations of neurons. Recent studies have demonstrated that neuronal death occurs in the nervous systems of vertebrates during certain periods of growth and development. However, the addition of soluble neuronal trophic factors from associated target tissues serves to mitigate this phenomenon of neuronal death. The following citations discuss neuronal trophic factors and their disclosures are hereby incorporated by reference: Chau, R. M. W., et al., Neuronotrophic Factor, 6 Chin. J. Neuroanatomy 129 (1990); Kuno, M., Target Dependence of Motoneuronal Survival: The Current Status, 9 Neurosci. Res. 155 (1990); Bard, Y. A., Trophic Factors and Neuronal Survival, 2 Neuron 1525 (1989); Oppenheim, R. W., The Neurotrophic Theory and Naturally Occurring Motoneuron Death, 12 TINS 252 (1989); Bard, Y. A., What, If Anything, is a Neurotrophic Factor?, 11 TINS 343 (1988); and Thoenen, H., and Edgar, D., Neurotrophic Factors, 229 Science 238 (1985).

In the vertebrate neuromuscular system, the survival of embryonic motoneurons have been found to be dependent upon specific trophic substances derived from the associated developing skeletal muscles. Skeletal muscles have been shown, by both in vivo and in vitro studies, to produce substances which are capable of enhancing the survival and development of motoneurons by preventing the embryonic motoneurons from degeneration and subsequent, natural cellular death. See O'Brian, R. J. and Fischbach, G. D., Isolation of Embryonic Chick Motoneurons and Their Survival In Vitro, 6 J. Neurosci. 3265 (1986); Hollyday, M. and Hamburger, V., Reduction of the Naturally Occurring Motor Neuron Loss by Enlargement of the Periphery, 170 J. Comp. Neurol. 311 (1976), whose disclosures are incorporated herein by reference. Similarly, several investigators have reported that chick and rat skeletal muscles possess certain trophic factors which can prevent the natural cellular death of embryonic motoneurons both in vivo and in vitro. See McManaman, J. L., et al., Purification of a Skeletal Muscle Polypeptide Which Stimulates Choline Acetyltransferase Activity in Cultured Spinal Cord Neurons, 263 J. Biol. Chem. 5890 (1988); Oppenheim, R. W., et al., Reduction of Naturally Occurring Motoneuron Death In Vitro by a Target Derived Neurotrophic Factor, 240 Science 919 (1988); and Smith, R. G., et al., Selective Effects of Skeletal Muscle Extract Fractions on Motoneurons Development In Vivo, 6 J. Neurosci. 439 (1986), whose disclosures are incorporated herein by reference.

In addition, a polypeptide has been isolated from rat skeletal muscle which has been found to selectively enhance the survival of embryonic chick motoneurons in vivo, as well the activity of choline acetyltransferase in these motoneurons. This polypeptide has been named Choline Acetyltransferase Development Factor (CDF) and its biological function has been demonstrated to be different from other trophic factors such as Nerve Growth Factor (NGF), Ciliary Ganglion Neurotrophic Factor (CNTF), Brain-Derived Neurotrophic Factor (BDNF), and Retinal Ganglion Neurotrophic Factor (RGNTF). See Levi-Montalcini, R., “Developmental Neurobiology and the Natural History of Nerve Growth Factor,” 5 Ann. Rev. Neurosci. 341 (1982); Varon, S., et al., Growth Factors. In: Advances in Neurology, Vol. 47: Functional Recovery in Neurological Disease, Waxman, S. G. (ed.), Raven Press, New York, pp. 493-521 (1988); Barde, Y. A., Trophic Factors and Neuronal Survival, 2 Neuron 1525 (1989); Chau, R. M. W., et al., The Effect of a 30 kD Protein from Tectal Extract of Rat on Cultured Retinal Neurons, 34 Science in China, Series B, 908 (1991), whose disclosures are incorporated herein by reference.

The isolation and characterization of two motoneuronotrophic factors from rat muscle tissue having apparent molecular weights of 35 kD and 22 kD has been reported. See Chau, R. M. W., et al., Muscle Neuronotrophic Factors Specific for Anterior Horn Motoneurons of Rat Spinal Cord. In: Recent Advances in Cellular and Molecular Biology, Vol. 5, Peeters Press, Leuven, Belgium, pp. 89-94 (1992). The 35 kD protein was defined as motoneuronotrophic factor 1 (MNTF1) and the apparent 22 kD protein as motoneuronotrophic factor 2 (MNTF2). These two trophic factors have been demonstrated in vitro to support the growth and/or regeneration of both isolated anterior horn motoneurons and spinal explants of rat lumber spinal cord.

Subsequently, the cloning and of human MNTF1 gene fragments from a human retinoblastoma cDNA library was reported. See U.S. Pat. No. 6,309,877. Human MNTF1 cDNA fragments were subcloned into expression vectors and the MNTF1 polypeptide fragments contained in the expressed fusion proteins exhibited biological activity similar to that of the “native” MNTF1 protein in that it supported the in vitro growth of rat anterior horn motoneurons.

Although various biological aspects of MNTF1 have been determined and a cDNA fragment encoding a peptide having MNTF activity has been reported, the full length cDNA for human MNTF1, was not disclosed. Thus, there remained a need to determine additional MNTF cDNA sequences, which can be used to map the entire MNTF gene within a specific region of human chromosomal DNA.

### SUMMARY OF THE INVENTION

The present invention is directed to nucleotide sequences encoding motoneuronotrophic factors, in particular polypeptides related to MNTF1, which map within chromosome 16q22, including MNTF associated polypeptides having the same upstream regulatory sequences, i.e., promoters and/or transcription start sequences as MNTF1. The present invention is also directed to novel DNA sequences, which include cDNAs encoding several open reading frames, including at least one motoneuronotrophic factor, human chromosomal DNA corresponding to the MNTF gene, to vectors which contain these novel DNA sequences, to expression systems and associated hosts which contain these novel DNA sequences, to synthetic MNTF peptides and the novel recombinant human MNTF1 proteins which are produced by the aforementioned expression systems. Additional aspects of the present invention include MNTF associated primers and probes for use in hybridization procedures, as well as compositions, kits and panels for use in such assays.

### DESCRIPTION OF THE FIGURES

The present invention may be better understood and its advantages appreciated by those individuals skilled in the relevant art by referring to the accompanying figures wherein:

FIG. 1 shows the expression pattern of the MNTF1 gene in the adult human MTC panel (Clontech);

FIG. 2 shows the expression pattern of the MNTF1 gene in the fetal MTC panel (Clontech):

FIG. 3 shows the analysis of total RNA isolated from six different human tissues, pituitary (lane 1), placenta (lane 2), brain (lane 3), retina (lane 4), heart (lane 5), fetal muscle (lane 6), by agarose gel electrophoresis;

FIG. 4 shows DNA fragments generated from a RACE procedure using total RNA from pituitary (lane 1), placenta (lane 2), fetal and adult brain (lane 3), retina (lane 4), heart (lane 5), and fetal muscle (lane 4);

FIG. 5 shows alignment of the MNTF cDNA from pituitary tissue with chromosome 16 sequences;

FIG. 6 verifies the variation of two positions of the cDNA compared to sequences on chromosome 16 by DNA sequencing;

### DESCRIPTION OF THE INVENTION

The present invention is related to a family of neuronotrophic factors, which possess the ability to exert a trophic and tropic effect on motoneurons, as well as the genes which encode these factors. It has been demonstrated that isolated factors, recombinant factors, and chemically synthesized polypeptide factors are capable of inducing the continued viability and neurite outgrowth of motoneurons. See, e.g., U.S. Pat. No. 6,309,877 and International PCT Patent Application filed Jan. 21, 2004 entitled “MNTF Peptides and Compositions and Methods of Use,” which claims the benefit of U.S. Provisional Application No. 60/441,772, filed Jan. 21, 2003, which are all incorporated herein by reference in their entirety. Therefore, these factors have been classified as “motoneuronotrophic factors” or “MNTFs.” Moreover, MNTFs exhibit anti-scar and anti-inflammatory effects, with further applications in the treatment of neuropathy, neuropathic pain, diabetic neuropathy and pain. The present invention includes new cDNA sequences isolated from human brain, pituitary and placenta tissue sources, as well as the corresponding DNA sequences from human chromosome 16.

For convenience, nucleic acids containing sequences encoding MNTF1 and/or additional flanking nucleic acid sequences of the full length MNTF1 gene, which maps on Chromosome 16 (SEQ ID NO:2), are referred to herein as a MNTF associated nucleic acids, polynucleotides, or oligonucleotides. Similarly, other polypeptides encoded by open reading frames found within the full length MNTF1 gene are referred to herein as MNTF associated proteins, polypeptide or peptides.

I. MNTF1 Biological Activity

MNTFs have been isolated from both rat and human sources and their biological activities have been examined both in vivo and in vitro. For example, their potential biological activity has been examined in both surgically-axotomized and hereditarily diseased animals. See, e.g., U.S. Pat. No. 6,309,877.

More recently, the trophic effect of MNTF was confirmed in vivo in studies showing that synthetic MNTF enhances peripheral nerve regeneration. Enclosed 8 mm gaps in the rat sciatic nerve were filled with 90% Vitrogen containing MNTF. The MNTF concentrations (Molar dilution) and resulting numbers of motoneurons crossing the gap to be labeled from the distal stump with Fluoro Gold at 1 month were: saline control, 540; 10⁻⁷ M, 678; 10⁻⁶ M, 765; 10⁻⁵ M, 873; 10⁻⁴ M, 1111, 10⁻³ M, 1130.

Moreover, the tropic effect of MNTF was also confirmed in recent in vivo studies. Transected and sutured rat femoral nerve were then bathed in MNTF at the optimal concentration of 10⁻⁴ M or in saline for 3 weeks. Regeneration was evaluated by double-labeling the femoral muscle and cutaneous branches (see Brushart, T. M. et al. The Journal of Neuroscience 22(14): 6631-6638, 2002, incorporated herein by reference, for methodology). After treatment with saline, a mean of 100 motoneurons projected correctly to muscle and a mean of 87 projected incorrectly to skin; a mean of 51 were double-labeled. After MNTF treatment, the mean number of motoneurons projecting correctly to muscle increased to 173 (p=0.0008), with means of 59 projecting to skin and 47 double-labeled. MNTF had no significant effect on the pattern of sensory neuron projection.

II. Expression and Cloning of MNTF Gene Sequences

Motoneuron Trophic Factor (MNTF) was originally isolated as a 33 amino acid peptide by screening a retinoblastoma cDNA library (Clontech) with antibodies to muscle extracts from 3 week old rats (U.S. Pat. No. 6,309,877, incorporated by reference herein). A 927 bp fragment of the MNTF gene was cloned and its nucleic acid sequence was determined (disclosed as SEQ ID NO:2 in U.S. Pat. No. 6,309,877).

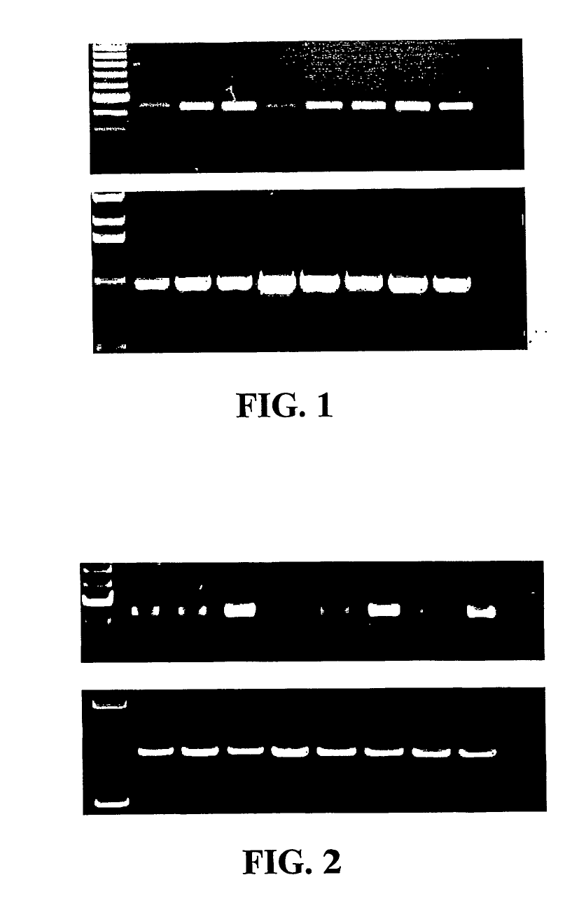

FIG. 1 shows that mRNAs containing MNTF encoding sequences are strongly expressed in the fetal thiymus, pituitary, liver, kidney, and in 8-9 week placenta, and are expressed weakly in fetal muscle. There is negligible expression in adult muscle.

Further cloning and sequencing experiments produced the following improvements in the MNTF sequence.

A partial sequence of brain cDNA included additional sequences upstream from the start of the 927 bp fragment (nucleic acid residues 1-582 of SEQ ID NO:1).

A relatively abundant cDNA of about 1.8 kb was isolated from pituitary tissue. The sequence was amplified by standard PCR and RACE and the sequence of the resulting 1859 bp cDNA was determined (see SEQ ID NO:1). SEQ ID NO:1 contains the same 5′ sequences upstream from the start of the 927 bp fragment as the partial brain cDNA sequence. In addition, SEQ ID NO:1 contains a portion that is an exact match to sequences 1-236 of the 927 bp fragment (nucleic acid residues 583-757 of SEQ ID NO:1). However, SEQ ID NO:1 contains a number of variations from the 927 bp fragment as summarized in Table 1.

TABLE 1

| 927 bp | | 1859 bp (SEQ ID NO: 1) | |
|---|---|---|---|
| Residue numbers | Sequence | Residue numbers | Sequence |
| 237-244 | nnnnnnnn | 758-765 | aaaaaaaa |
| 365-371 | nnnnnnn | 886-892 | aaaaaaa |
| 583-587 | tgatc | 1104-1108 | gatca |
| 603-604 | a--g | 1126-1129 | aagg |
| 640-646 | --tcaggtc | 1165-1173 | catgaggtc |
| 647-653 | agaa--gct | 1174-1182 | agaaaagct |
| none | none | 1183-1192 | ccaatgata |
| 654-658 | -ccgaa-- | 1192-1198 | tccgagg |
| 684-685 | tc | 1224-1225 | ct |
| 727 | g | 1267 | c |
| 749 | t | 1289 | a |
| 781-782 | c-a | 1321-1323 | caa |
| 793-794 | c-a | 1334-1336 | caa |
| 797-798 | g-a | 1139-1141 | gaa |
| 803-804 | a--c | 1346-1349 | aacc |
| 819-827 | nnnnnnnnn | 1364-1372 | ggggggggg |
| 858-861 | acac | 1403-1406 | caca |
| 905-906 | a-c | 1450-1452 | acc |
| 921-927 | cggaatt | 1467-1473 | aatctt |

Accordingly, nucleic acid residues 758-1473 of SEQ ID NO:1 are an inexact match to corresponding regions of the originally disclosed 927 bp sequence. Moreover, the sequence of additional downstream residues of the MNTF transcript, i.e. residue numbers 1474-1859 of SEQ ID NO:1 have not been previously disclosed.

A cDNA library, prepared from normalized human placenta, was also utilized to isolate and sequence another MNTF cDNA from another tissue source. The sequence of the placenta cDNA sequence corresponds to SEQ ID NO:1

Accordingly the present invention provides improved sequence information for MNTF encoding and/or MNTF associated nucleic acids, namely SEQ ID NO:1, sequences complementary to SEQ ID NO:1 and portions or fragments thereof. Moreover, preferred embodiments will include portions of SEQ ID NO:1 (other than nucleic acid residues 758-1473 of SEQ ID NO:1) that do not entirely correspond to portions of the previously disclosed 927 bp fragment.

III. MNTF Gene Sequences on Chromosomal DNA

To compare a nucleotide or polypeptide sequence with the corresponding SEQ ID NO: 1 sequence, a global alignment of the sequences can be performed using the BLAST programs publicly available through the National Center for Biotechnology Information (on the World Wide Web at ncbi.nlm.nih.gov). Prior to performing a global alignment, SEQ ID NO: 1 can be submitted to GenBank. Default parameters provided by the National Center for Biotechnology Information can be used for a global alignment.

DNA analysis using a database, such as htt://www.ncbi.nlm.nih.gov or http://genome.ucsc.edu, shows the gene containing SEQ ID NO:1 lies on chromosome 16, Band: 16q22. The chromosome band track represents the approximate location of bands seen on Giemsa-stained chromosomes at an 800 band resolution. Barbara Trask, Vivian Cheung, Norma Nowak and the rest of the BAC Resource Consortium used fluorescent in-situ hybridization (FISH) to determine a cytogenetic location for large genomic clones on the chromosomes. For more information about the BAC Resource Consortium, please see “Integration of cytogenetic landmarks into the draft sequence of the human genome”, Nature, 409:953-958, February 2001 and the accompanying web site Human BAC Resource.

In Genbank, the locus is AC092383, Accession # AP001588, Homo sapiens chromosome 16 clone RP11-787D11. The position of SEQ ID NO:1 in clone RP11-787D11 is between 80244 and 80730.

The MNTF 1859 bp cDNA sequence (SEQ ID NO:1) maps within an intron of the NIN283 gene, located from 74813548 to 74907022 (using UCSC genome blat) of chromosome 16q22. To determine the location of any upstream regulatory sequences before the MNTF cDNA sequence on Chromosome 16, theoretical modeling with computer analysis was performed to identify potential promoter and/or transcription start sites with the region from 74813548 to 74907022 (using UCSC genome blat) of chromosome 16q22.

SEQ ID NO:2 shows a 4359 bp sequence from chromosome 16 (corresponding to 74818596 to 74814238 using UCSC genome blat) which includes genomic sequences upstream from the MNTF cDNA sequences, including a putative First Promoter Site (nucleic acid residues 862-911 of SEQ ID NO:2), a putative Second Promoter Site (nucleic acid residues 2315-2364 of SEQ ID NO:2), and a potential transcription start sequence (nucleic acid residues 2355-2501 of SEQ ID NO:2). SEQ ID NO:2 also includes MNTF cDNA (nucleic acid residues 2501-4359), which contains a region (nucleic acid residues 3058-3120) encoding a 21 amino acid peptide (SEQ ID NO:29) known to have MNTF activity.

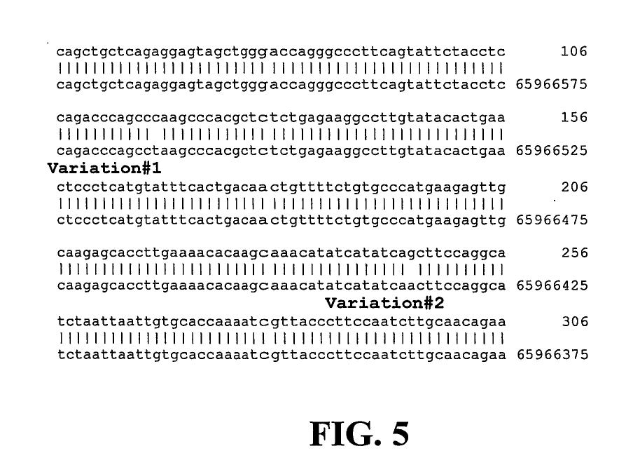

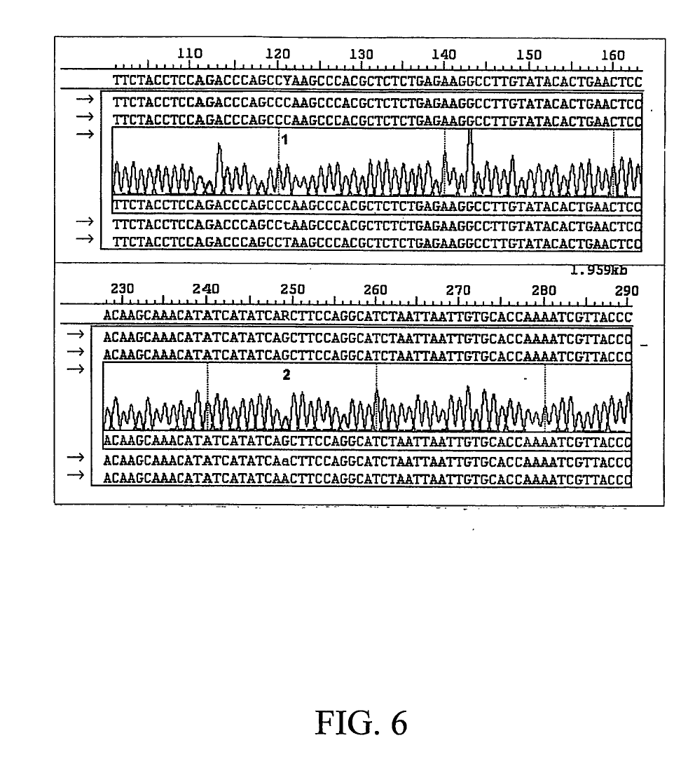

FIG. 5 shows alignment of the MNTF cDNA from pituitary tissue with chromosome 16 sequences. The MNTF cDNA sequences differed from the chromosome 16 sequences at two positions. FIG. 6 verifies the variation at two positions of the cDNA sequence, compared to sequences on chromosome 16, by DNA sequencing:

Accordingly additional embodiments of the present invention include SEQ ID NO:2, sequences complementary to SEQ ID NO:2 and portions or fragments thereof. Preferred fragments will include at least one putative promoter and/or the potential start sequence.

IV. MNTF Associated Polypeptides

Analysis of the three reading frames derived from the cDNA sequences shows there are several open reading frames (ORFs) within the MNTF cDNA sequence (SEQ ID NO:1). The amino acid sequences shown below in Table 2 are putative peptide sequences encoded in each reading frame, which begin with an initiator codon (ATG) for methionine (M) and end at a termination codon.

TABLE 2

| | |
|---|---|
| SEQ | No. |

| | | |
|---|---|---|
| ID | of | SEQ ID |

| | | | | |
|---|---|---|---|---|
| NO. | Name | a. a. | amino acid sequence | NO:1 ORF |

| | | | | |
|---|---|---|---|---|
| 13 | RF #1 | 24 | MSWKIPWSVSGEMEPMLHTKIHLK | 337-408 |
| | (1) | | | |
| 14 | RF #1 | 56 | MLDVLQKDMMVLHSKEWITYNINS | 580-747 |
| | (2) | | SLPYTLLTPFPKGLICSNLPVPTV | |
| | | | QWLSLPSP | |
| 15 | RF #1 | 78 | MEPGSSIINKFLVRGLRNFSKKST | 826-1059 |
| | (3) | | PFLPPYISHMFFNTKNSVILEKLL | |
| | | | TVLLSSKPDAHYSSFKHQTSHLKN | |
| | | | MANSLD | |
| 16 | RF #1 | 30 | MSCFSLRAEFHEVRKAPMISGGLS | 1135-1224 |
| | (4) | | EIKISS | |
| 17 | RF #1 | 21 | MPHRDRLPVTKRCRFTGRPST | 1774-1836 |
| | (5) | | | |
| 18 | RF #2 | 21 | MKSCKSTLKTQANISYQLPGI | 140-202 |
| | (1) | | | |
| 19 | RF #2 | 48 | MCNRGNRCPGRSHGLFQVRWNQCC | 317-460 |
| | (2) | | TRRFISNEMHRKSQLLYVNKRINP | |
| 20 | RF #2 | 6 | MSCRRT | 587-604 |
| | (3) | | | |
| 21 | RF #2 | 30 | MKKNKVSQLKSEILESNYKLITVA | 755-844 |
| | (4) | | WNLVVA | |
| 22 | RF #2 | 29 | MLITVLLNTKLVISKIWLTLWTKF | 995-1080 |
| | (5) | | HRKNY | |
| 23 | RF #2 | 7 | MRSEKLQ | 1166-1186 |
| | (6) | | | |
| 24 | RF #2 | 25 | MKTNPRLFAGGGKEIPQKSTLFHS | 1337-1411 |
| | (7) | | S | |
| 25 | RF #2 | 38 | MPDLADRPVFLLPGLFCPPARSLGN | 1592-1705 |
| | (8) | | PPPTFCWPVTRSS | |
| 26 | RF #3 | 11 | MYFTDNCFLCP | 108-141 |
| | (1) | | | |
| 27 | RF #3 | 5 | MVCFR | 354-368 |
| | (2) | | | |
| 28 | RF #3 | 11 | MKCTGKANYFM | 408-440 |
| | (3) | | | |
| 29 | RF #3 | 21 | MLSAFSRYARCLAEGHDGPTQ | 538-621 |
| | (4) | | | |
| 30 | RF #3 | 20 | MLQPSCPHSSVALPTLTMIG | 696-755 |
| | (5) | | | |
| 31 | RF #3 | 25 | MPNFDQMPERAKGNHVLLLTQGRV | 1092-1166 |
| | (6) | | P | |
| 32 | RF #3 | 25 | MNSSRFSESSFSPVMCQQPGNNAP | 1710-1784 |
| | (7) | | P | |

SEQ ID NO: 29 includes 21 of the 33 amino acids disclosed as SEQ ID NO:4 in U.S. Pat. No. 6,309,877, which has been shown to have MNTF activity. Interestingly, SEQ ID NO: 29 is not encoded by the first open reading frame of the MNTF and mRNA. There does not appear to be a clear Kozak sequence before any of the putative peptide regions, suggesting the presence of addition ORFs upstream from the MNTF encoding sequence may provide an alternative mechanism for translational control on expression of MNTF and/or MNTF associated polypeptides.

Additional embodiments include biologically active mammalian polypeptides, including for example, those that are isolated, expressed in vitro, or chemically synthesized, that comprise an amino acid sequence which is at least 80% identical to the amino acid sequence(s) disclosed herein and methods of using the polypeptide to promote the survival, growth, proliferation, or maintenance of mammalian neurons and the differentiation of neural stem cells into neurons. In alternative embodiments, the invention includes polypeptides comprising at least 10 consecutive amino acid residues, at least 15 consecutive amino acid, at least 20 consecutive amino acid residues, at least 25 consecutive amino acid residues, or at least 30 consecutive amino acid residues of the putative peptide sequences disclosed herein.

An MNTF associated polypeptide within the scope of this invention can also be a fusion protein containing an open reading frame of SEQ ID NO:1 attached to a heterologous protein. A heterologous protein has an amino acid sequence not substantially similar to the MNTF associated polypeptide. The heterologous protein can be fused to the N-terminus or C-terminus of the MNTF associated polypeptide. Fusion proteins can include, but are not limited to, enzymatic fusion proteins, for example beta-galactosidase fusions, poly-His fusions, MYC-tagged fusions and Ig fusions. Such fusions proteins, particularly poly-His fusions, can facilitate the purification of recombinant MNTF associated polypeptides.

A fusion protein can be produced by standard recombinant DNA techniques. For example, PCR amplification of gene fragments can be carried out using anchor primers which give rise to complementary overhangs between two consecutive gene fragments. The fragments can be annealed and re-amplified to generate a chimeric gene sequence (Ausubel et al., Current Protocols in Molecular Biology, 1992). The chimeric gene can be expressed in an appropriate host cell. Alternatively, a DNA fragment encoding a MNTF associated polypeptide can be cloned in a commercially available expression vector that already contains a heterologous protein, with the result being a MNTF associated polypeptide fused in-frame to the heterologus protein.

V. Expression Vectors

Another version of the present invention provides a vector containing a MNTF associated polynucleotide encoding one or more of the open reading frame sequences described herein. The vector can be a cloning vector for maintaining nucleic acid molecules, or an expression vector. A variety of cloning and expression vectors are well known to those of skill in the art. Examples include plasmid vectors, single or double stranded phage vectors, single or double stranded DNA or RNA viral vectors, or artificial chromosomes, such as BAC and YAC. An expression vector contains a nucleotide sequence encoding a MNTF associated polypeptide as described herein operably linked to a promoter. Promoters, terminators and other regulatory regions suitable for controlling transcription and translation in a variety of prokaryotic and eukaryotic host cells are well known in the art (Sambrook et al., Molecular Cloning: A Laboratory Manual. 2nd ed., Cold Spring Harbor Laboratory Press, Cold Spring Harbor, N.Y., 1989).

Yet another version of the invention further provides a host cell containing an expression vector of this invention. The host cell can be a mammalian cell, plant cell, insect cell, yeast and other fungi, or bacteria. Suitable host cells for various expression vectors are well known to those of skill in the art (Sambrook et al., Molecular Cloning: A Laboratory Manual. 2nd ed., Cold Spring Harbor Laboratory Press, Cold Spring Harbor, N.Y., 1989).

An expression vector can be introduced into a suitable host cell by techniques such as calcium phosphate transfection, DEAE-dextran-mediated transfection, cationic lipid-mediated transfection, electroporation, transduction, infection, lipofection, and other techniques well known to those in the art (Sambrook, et al. Molecular Cloning: A Laboratory Manual. 2nd, ed., Cold Spring Harbor Laboratory, Cold Spring Harbor Laboratory Press, Cold Spring Harbor, N.Y., 1989).

VI. Nucleic Acid Detection

In addition to their use in directing the expression of MNTF associated proteins, polypeptides and/or peptides, the nucleic acid sequences disclosed herein have a variety of other uses. For example, they have utility as probes or primers for embodiments involving nucleic acid hybridization. Thus, another aspect of the invention includes alternative methods for detecting the expression of MNTF associated transcripts by detecting the hybridization of nucleotide sequences in a patient's biological sample with the nucleotide sequences coding for MNTF protein or its derivatives.

Nucleotide hybridization assays may be used, whereby nucleic acids from a patient's biological sample are contacted to the MNTF associated polynucleotides of the present invention under hybridization conditions, and the hybridization products are detected. This method could be used to detect MNTF associated genomic DNA or mRNA. Northern Blot analysis, RT-PCR or PCR and ligase chain reaction (LCR) may be used as the basis of the assay, these techniques are known to those skilled in the art. PCR and LCR techniques are widely available in the art. For example, the basic PCR techniques are described in U.S. Pat. Nos. 4,683,202; 4,683,195; 4,800,159; and 4,965,188. The basic LCR techniques are described in EPA-320,308; EPA-439,182; EPA-336,731; WO 89/09835; WO 89/12696, and WO 90/01069.

Oligonucleotide probes or primers preferably contain at least 10 contiguous nucleotides or at least 30 contiguous nucleotides with at least 60% homology along the length of the MNTF associated nucleotide sequence being compared. Examples of such probes/primers and methods for conducting the PCR for detection of MNTF associated nucleic acids are described in Examples 1 and 3.

Accordingly, the nucleotide sequences of the invention may be used for their ability to selectively form duplex molecules with complementary stretches of DNAs and/or RNAs or to provide primers for amplification of DNA or RNA from samples. Table 3 presents a number of exemplary MNTF associated nucleotide sequences that can be utilized as probes and/or primers, as described in further detail in Examples 1 and 3.

TABLE 3

| | | |
|---|---|---|
| SEQ | No. | SEQ ID |
| ID | of | No. 1 |

| | | | | |
|---|---|---|---|---|
| NO. | Name | nt | Sequence | alignment |

| | | | | |
|---|---|---|---|---|
| 3 | Primer | 22 | TTTCTTCCTCCCTACATCTCTC | 901-922 |
| | F901 | | | |
| 4 | Primer | 21 | GAGGGTAATATCTGTTGGATC | 1441-1421 |
| | R1441 | | | |
| 5 | Primer | 20 | TTGGGGACATTTTGGGGTGA | 522-541 |
| | F522 | | | |
| 6 | Primer | 20 | GCTCGATGTCTTGCAGAAGG | 582-601 |
| | F582 | | | |
| 7 | Primer | 21 | AGGGTAACACTTAGAAGTAGC | 1258-1238 |
| | R1258 | | | |
| 8 | Primer | 19 | TCACCCCAAAATGTCCCCA | 541-523 |
| | R541 | | | |
| 9 | Vector | 27 | CTGTTAGCTTGGTACCGAGCTCGGATC | none |
| | Primer | | | |
| 10 | Primer | 27 | TAGGGGAAAGATTGCTCCTGCCTTTAG | 1479-1453 |
| | F1479 | | | |
| 11 | Primer | 27 | TATTGCCTGGCTGTTGGCACATGACTG | 1773-1747 |
| | R1773 | | | |
| 12 | Primer | 27 | CTGCTCCATGCTAAGTGCTTGGTCTTC | 1849-1823 |
| | R1849 | | | |

Depending on the application envisioned, one would desire to employ varying conditions of hybridization to achieve varying degrees of selectivity of the probe or primers for the target sequence.

For applications requiring high selectivity, one will typically employ relatively high stringency conditions to form the hybrids. For example, relatively low salt and/or high temperatures conditions, such as provided by about 0.02 M to about 0.10 M NaCl at temperatures of about 50° C. to about 70° C. Such high stringency conditions tolerate little, if any, mismatch between probe or primers and the template or target strand and would be particulary suitable for isolating specific genes or detecting specific mRNA transcripts. It is generally appreciated that conditions can be rendered more stringent by the addition of increasing amounts of formamide.

For other applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less strigent by increasing salt concentration and/or decreasing temperature.

In certain embodiments, it will be advantageous to employ nucleic acids of defined sequences of the present invention in combination with an appropriate means, such as a label for determining hybridization. A wide variety of indicator means are known in the art, including fluorescent, radioactive, enzymatic or other ligands, such as avidin/biotin, which are capable of being detected.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA or RNA is adsorbed or otherwise affixed to a selected matrix or surface. This fixed, single stranded nucleic acid is then subjected to hybridization with selected probes under desired conditions. Optimization of hybridization conditions for the particular application are well known to those of skill in the art. After washing the hybridized molecules to remove non-specifically bound probe molecules, hybridization is detected and/or quantified, by determining the amount of bound label.

Nucleic acids used as a template for amplification may be isolated from cells, tissues or other samples according to standard methodologies (Sambrook et al., 1989). In certain embodiments, anaysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA into a complementary DNA.

The term “primer” as used herein, is meant to encompass any nucleic acid that is capable of priming the synthesis of a nascent nucleic acid in a template-dependent process. Typically primers are oligonucleotides from ten to twenty and/or thirty base pairs in length, but longer sequences can be employed.

Pairs of primers designed to selectively hybridize to nucleic acids corresponding to portions of SEQ ID NO:1 or SEQ ID NO:2 are contacted with the template nucleic acids that permit selective hybridization. Once hybridized, the template-primer complex is contacted with one or more enzymes that facilitate template-dependent nucleic acid synthesis. Multiple rounds of amplification, also referred to as cycles, are conducted until a sufficient amount of amplification product is produced.

The amplification product may be detected or quantified. In certain applications, the detection may be performed by visual means. Alternatively, the detection may involve indirect detection of the product via chemiluminescence, radioactive scintigraphy of incorporated radiolabel or fluorescent label or even via a system using electrical and/or thermal impulse signals (Affymax technology, Bellus, 1994).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separation of nucleic acids may also be effected by chromatographic techniques known in the art, such as adsorption, partition, ion-exchange, hydroxylapatite, molecular sieve, reverse-phas, column, paper, thin-layer and gas chromatography as well as HPLC.

VII. Assay Kits, Reagents and Panels

Also included are assay kits for nucleotide hybridization assays which include probes and/or primers which are specific for MNTF associated nucleic acids or their derivatives. The kits may also include sample preparation reagents, wash reagents, detection reagents and signal producing reagents.

### EXAMPLE 1

To identify which tissues had the highest expression of MNTF, PCR products were amplified from cDNAs obtained from a number of different tissue sources. Adult and fetal cDNA panels derived from various tissues were ordered (Clontech) and gene specific primers were synthesized based on chromosomal mapping experiments (see below)

The following primer set was used:

| | |
|---|---|
| Forward: | |
| 5′ TTT CTT CCT CCC TAC ATC TCT C 3′ | (SEQ ID NO:3) |
| Reverse: | |
| 5′′ GAG GGT AAT ATC TGT TGG ATC 3′ | (SEQ ID NO:4) |

The expected PCR product was 541 base pairs in length.

A. PCR Protocol:
1. Master Mix PCR 1×

35 μl water

5 μl 10× buffer

1.5 μl MgCl₂ (50 mmol)

1 μl DNTP

1 μl each primer

2U Taq Platinum polymerase enzyme

5 μl DNA template
2. PCR Cycling

5′ 95° C.

38 cycles:

| | |
|---|---|
| 30″ | 95° C. |
| 30″ | 53° C. annealing |
| 45″ | 72° C. elongation |
| 5′ | 72° C. final elongation |

B. Results

FIG. 1 shows that MNTF adult tissue expression was about the same in all tissues except brain and skeletal muscle, where it was lower. Referring to FIG. 1, primers were used to amplify a 541 bp product of MNTF1 (top panel), which was normalized against the G3PDH housekeeping gene expression (bottom panel). Samples in the lanes are consistent for both gels. From left to right: lane 1, marker: lane 2, brain: lane 3, lung; lane 4, thymus; lane 5, skeletal muscle; lane 6, kidney; lane 7, heart; lane 8, liver; lane 9, placenta; and lane 10, negative H₂O control.

FIG. 2 shows that MNTF fetal tissue expression was highest in thymus, kidney, and liver and was lowest in skeletal muscle. Referring to the top panel of FIG. 2, lane 1, marker: lane 2, brain: lane 3, lung; lane 4, thymus; lane 5, skeletal muscle; lane 6, spleen; lane 7, kidney; lane 8, heart; lane 9, liver; and lane 10, negative H₂O control.

This experiment determined that thymus, kidney and liver tissues have relatively high levels of MNTF expression in both fetuses and adults.

### EXAMPLE 2

The total RNA was extracted by RISO RNA isolation method (Cat#06-200) (Genemed Biotechnologies, Inc., South San Francisco, Calif.) from the following tissues: Hippocampus, placenta, brain (both fetal brain and adult brain), retina, pituitary, heart, fetal muscle The method combines potent known inhibitors of RNases, guanidine thiocyanate (GTC) and beta-mercaptoethanol to slow the rate of RNA degradation. It also disrupts nucleoprotein complexes allowing RNA to be released into solution and isolated free from protein contamination. The final total RNA is further purified from contaminants by acid phenol and chloroform extraction and concentrated by precipitation with isopropanol. Further purification was done by an ethanol precipitation and wash to remove any residual protein and contaminating salts.

The purity and amount of total RNA extracted from the tissue samples was determined by gel electrophoresis and indicated by A260/A280 ratios. About 3.5 mg of total RNA was isolated from one gram of tissue; the A260/A280 ratio was 1.65 and the A260/A230 ratio was about 1.20.

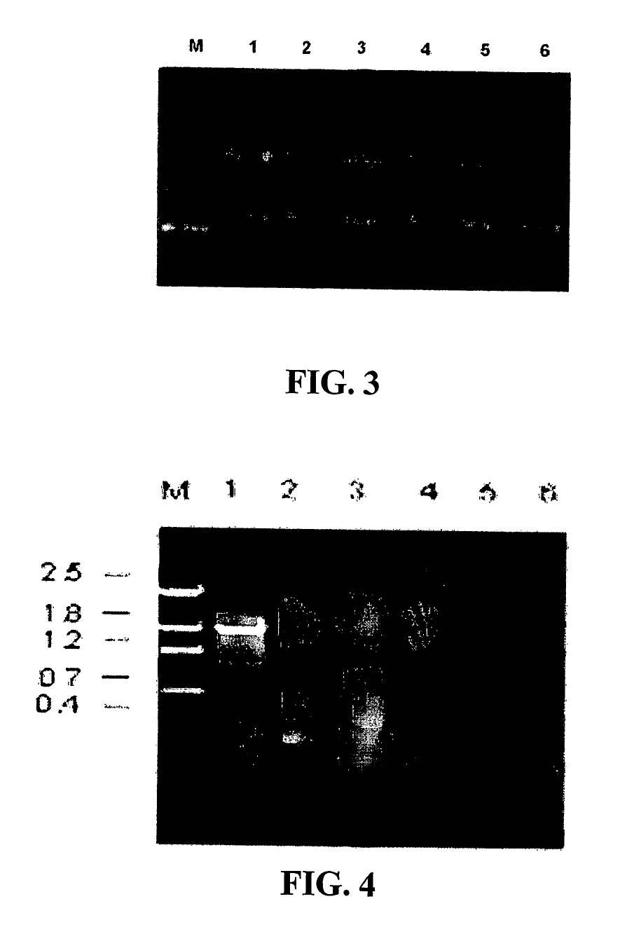

FIG. 3 shows an analysis of total RNA isolated from 10 mg samples of different human tissues. Three micrograms of RNA were loaded per lane on a 1.2% agarose formaldehyde gel. Lanes 1-6 show total RNA from pituitary, placenta, brain, retina, heart, and fetal muscle, respectively. Lane M shows a 0.24-9.7 kb RNA ladder.

The cDNA was prepared with Genemed cDNA preparation technology (patent pending) from mRNA. Some of the mRNA was purchased from Clontech (Palo Alto, Calif.). A representative cDNA library was constructed by enzymes involved in copying mRNA into double-stranded cDNA and subsequently preparing the termini for vector ligation. The first strand was synthesized by Avian Myeloblastosis Virus Reverse Transcriptase and Random Hexameric Primers. The second strand was synthesized using RNase H and DNA Polymerase I. After treatment with T4 DNA Polymerase to flush the ends, the double-stranded cDNA molecules was prepared for cloning by adaptor ligation.

### EXAMPLE 3

DNA was prepared for sequencing using a standard DNA purification kit. DNA Sequencing was performed using the dideoxy method developed by Sanger et al. (1977), which takes advantage of the ability of DNA polymerase to incorporate 2′,3′-dideoxynucleotides as substrates. Single-stranded DNA template is copied with DNA polymerase by adding nucleotides to an extension chain. Chain elongation occurs at the 3′ end of a primer, an oligonucleotide that anneals to the template. The deoxynucleotide added to the extension product is selected by base-pair matching to the template. The extension product grows by the formation of a phosphodiester bridge between the 3′-hydroxyl group at the growing end of the primer and the 5′-phosphate group of the incoming deoxynucleotide. Growth is in the 5′ to 3′ direction. DNA polymerases can also incorporate analogues of nucleotide bases. When a dideoxynucleotide analogue is incorporated at the 3′ end of the growing chain, chain elongation is terminated selectively at A, C, G, or T because the chain lacks a 3′-hydroxyl group.

An ABI 377 sequencer from PE Applied Biosystems was utilized for the sequencing process. In the Applied Biosystems strategy for automated fluorescent sequencing, fluorescent dye labels are incorporated into DNA extension products using 5′-dye labeled primers (dye primers) or 3′-dye labeled dideoxynucleotide triphosphates (dye terminators). The DNA sequencers detect fluorescence from four different dyes that are used to identify the A, C, G, and T extension reactions. Each dye emits light at a different wavelength when excited by an argon ion laser and four colors indicating four bases can be detected and distinguished in a gel.

### EXAMPLE 3

Two different strategies, standard PCR and 5′-RACE, have been used for isolation and screening of the MNTF genes.

A. Standard PCR

The first strategy is to screen the gene with different primer sets. The purpose is to find if a full length MNTF sequence exists in the targeted cDNA library. The following oligonucleotide sequences are examples of primer pairs for use in standard PCR amplification of MNTF sequences.

| | |
|---|---|
| TTGGGGACATTTTGGGGTGA | (SEQ ID NO:5) |
| GCTCGATGTCTTGCAGAAGG | (SEQ ID NO:6) |
| AGGGTAACACTTAGAAGTAGC | (SEQ ID NO:7) |
| TCACCCCAAAATGTCCCCA | (SEQ ID NO:8) |

A Standard PCR method was used for screening cDNA prepared from different tissues. Each pair of primers was used to amplify a cDNA library from hippocampus, fetal brain, placenta, retina, pituitary, fetal muscle, or heart tissue.

Amplification conditions consisted of 20 mM Tris-HCl, pH 7.5, 50 mM KCl, 3.5 mM MgCl₂, 0.1% Triton X-100, 0.8 mM dNTPs, 100 mol of each primer and 2.5 u Taq DNA polymerase from Applied Biosystems. Thirty-five cycles were performed as follows: 94° C., 1 minute; 55° C., 2 minutes; 72° C., 3 minutes.

DNA amplification products were separated by electrophoresis and viewed under UV. When there was a visibly amplified band, it was isolated on a low melting agarose gel, phenol extracted and ethanol precipitated. The amplified bands were prepared for cloning. One partial PCR fragment,was isolated and sequenced. A partial DNA sequence, about 700 base pairs in length, corresponding to nucleic acid residues 1-713 of SEQ ID NO:1 amplified from cDNA prepared from brain tissue.

B. 5′-RACE Amplification

5′-RACE is another strategy used to find the 5′-end of the MNTF gene. 5′ RACE or anchored PCR facilitates the isolation and characterization of MNTF 5′ ends from messenger RNA (mRNA). The RACE System for rapid amplification of cDNA ends from Invitrogen Life Technologies (Carlsbad, Calif.) was modified and used to obtain the MNTF cDNA. The RACE procedure was used to amplify cDNA sequences from total mRNA between a defined internal site and unknown sequences at either the 3′ or the 5′-end of the MNTF mRNA. After isolating mRNA from different tissues, first strand cDNA synthesis is primed using a gene-specific antisense oligonucleotide. This permits cDNA conversion of MNTF specific mRNA, and related families of mRNAs, and maximizes the potential for complete extension to the 5′-end of the message. Following cDNA synthesis, the first strand product is purified from unincorporated dNTPs and MNTF primer. TdT (Terminal deoxynucleotidyl transferase) is used to add homopolymeric tails to the 3′ ends of the cDNA. The tailed cDNA is then amplified by PCR using a mixture of a nested gene-specific primer, and a combination of a complementary homopolymer-containing anchor primer and corresponding adapter primer which permit amplification from the homopolymeric tail. This allows amplification of the unknown sequences between the MNTF specific primer sequence and the 5′-end of the mRNA.

FIG. 4 shows a DNA fragment generated from the RACE procedure. About 0.1 μg of mRNA was used for the reaction. The result shown in FIG. 4 was obtained by using 1 μl of the RACE reaction. The reaction temperatures were 94° C. for 2 min; 30 cycles of 94° C. for 50 seconds, 65° C. for 30 seconds, and 72° C. for 3 minutes; and finally 72° C. for 10 minutes. Lanes 1-6 of FIG. 4 show total RNA from pituitary, placenta, brain (both fetal brain and adult brain), retina, heart, and fetal muscle, respectively. A DNA molecular weight marker is labeled as M. A relatively abundant RACE product of about 1.8 kb was generated from pituitary RNA.

The following oligonucletide sequences can be used as primers for amplifying MNTF sequences using the RACE procedure:

| | |
|---|---|
| 5′-CTG TTA GCT TGG TAC CGA GCT CGG | (SEQ ID NO:9) |
| ATC-3′ | |
| 5′-TAG GGG AAA GAT TGC TCC TGC CTT | (SEQ ID NO:10) |
| TAG-3′ | |
| 5′-TAT TGC CTG GCT GTT GGC ACA TGA | (SEQ ID NO:11) |
| CTG-3′ | |
| 5′-CTG CTC CAT GCT AAG TGC TTG GTC | (SEQ ID NO:12) |
| TTC-3′ | |

A cDNA for the MNTF gene was isolated from pituitary tissue by RACE. The sequence shown in SEQ ID NO:1 was assembled from several DNA sequencing runs. The sequence comprised 1859 contiguous bases corresponding to the MNTF mRNA, with an average length per sequencing run of 919 bases.

### EXAMPLE 4

A Superscript cDNA library, prepared from normalized human placenta, 8-9 weeks, was purchased from Invitrogen (see Catalog # SL.2NBHP89W)ln order to facilitate the amplification and cloning of the MNTF gene from the 5′-cDNA end and 3′- cDNA ends, a polymerase chain reaction-based RACE technique and protocol was used.

MNTF cDNA was isolated from the cDNA library. The 5′-end sequence was identified using 5′-end RACE and the 3′-end sequence was identified using 3′-end RACE. The method of isolating the gene was performed according to the manual of the DNA RACE kit. The details of steps are described by the manufacturer: Ambion, Inc., Austin, Tex. The isolated gene was then sequenced for gene identity confirmation.

MNTF cDNA was subcloned into a vector, pcDNA3.1/V5-His-TOPO vector. The junction of both 5′-end and 3′-end sequences were confirmed with DNA sequencing reactions.

The entire DNA sequence of the placenta cDNA corresponds to the MNTF associated cDNA from pituitary (SEQ ID NO:1).

### EXAMPLE 5

A gene identified as NIN283 also lies on chromosome 16q22. NIN283 is expressed in response to nerve injury (Accession # NM 032268, a 4633 bp mRNA sequence). The published gene sequence of NIN283 is located on chromosome 16q22 from 74812469 to 74813547, then from 74907023 to 74907118, 74918235 to 75918340, 74919933 to 7490022, and 74921184 to 74924445. The MNTF 1859 bp cDNA sequence lies on chromosome 16q22 from 74814238 to 74816096 (using UCSC genome blat). There is no overlap or match of the two nucleotide sequences, although the MNTF gene sequence lies between the first and second fragment (or exon), within an intron region of the NIN283 gene.

To ensure that the MNTF gene was in fact independent of NIN283, and not an exon of NIN283 in an undescribed spliceform, an RT-PCR was performed using poly A(+) RNA from skeletal muscle. Three sets of primers were created—one with the forward primer in the first exon of NIN283 and the reverse primer in the coding region of MNTF and two with forward primers in the coding region of MNTF and reverse primers in within exon 4 of NIN283. If a splice form existed with MNTF as an exon of NIN283, one would expect to see a PCR product. However, no significant PCR products were formed in any of the PCR reactions. The results of these RT-PCR reactions make it highly unlikely that MNTF and NIN283 are related.

### Additional Embodiments

Although the present invention has been described in considerable detail with reference to certain preferred versions thereof, other versions are possible. For example, tissue-specific and/or developmental stage variations in transcription start site usage of mRNA transcripts have been described by others. Accordingly, the 5′ end of MNTF associated mRNA or cDNA may include additional or fewer nucleic acid residues, depending on the tissue, cell or developmental stage, that may include selected portions of SEQ ID NO:2, e.g. the putative transcription start site. Moreover, corresponding MNTF associated oligonucleotides used in hybridization and/or amplification assays, in accordance with aspects of the present invention, would have utility providing a more definitive expression profile of such variations. Therefore, the spirit and scope of the present invention, as characterized in the appended claims, should not be limited to the description of the preferred versions contained therein.

1. An isolated MNTF associated polynucleotide selected from the group consisting of a sequence comprising SEQ ID NO:1; a sequence comprising SEQ ID NO:2; and complements of the preceding.

2. The isolated polynucleotide of claim 1 comprising a fragment of SEQ ID NO:1, said fragment comprising i) a 5′ terminus selected from residues 1-1849 of SEQ ID NO1, and ii) at least ten consecutive nucleic acid residues of SEQ ID NO:1 including the 5′ terminus and a 3′ terminus, wherein the 3′ terminus of said fragment is selected from residues 10-1859 of SEQ ID NO:1.

3. An isolated polynucleotide comprising a fragment of SEQ ID NO:1, provided that the nucleotide sequence of said fragment contains at least one nucleotide selected from any of the following: residues 1-582; residues 758-765; residues 886-892; residues 1104-1108; residues 1126-1129; residues 1165-1173; residues 1174-1182; residues 1183-1192; residues 1192-1198; residues 1224-1225; residue 1267; residue 1289; residues 1321-1323; residues 1334-1336; residues 1339-1341; residues 1346-1349; residues 1364-1372; residues 1403-1406; residues 1450-1452; residues 1467-1473, and residues 1474-1859 of SEQ ID NO:1.

4. The isolated polynucleotide of claim 1 comprising a fragment of SEQ ID NO:1 selected from the group consisting of:
a) SEQ ID NO:3;
b) SEQ ID NO:5;
c) SEQ ID NO:6; and
d) SEQ ID NO:10.

5. The isolated polynucleotide of claim 1 comprising a fragment having a nucleic acid sequence fully complementary SEQ ID NO:1 that is selected from the group consisting of:
a) SEQ ID NO:4;
b) SEQ ID NO:7;
c) SEQ ID NO:8;
d) SEQ ID NO:11; and
e) SEQ ID NO:12.

6. The isolated polynucleotide fragment of claim 3 comprising at least one open reading frame.

7. The isolated polynucleotide of claim 6 wherein the at least one open reading frame encodes a polypeptide selected from the group consisting of:
a) SEQ ID NO:13;
b) SEQ ID NO:14;
c) SEQ ID NO:15;
d) SEQ ID NO:16;
e) SEQ ID NO:17;
f) SEQ ID NO:18;
g) SEQ ID NO:19;
h) SEQ ID NO:20;
i) SEQ ID NO:21;
a) SEQ ID NO:22;
b) SEQ ID NO:23;
c) SEQ ID NO:24;
d) SEQ ID NO:25;
e) SEQ ID NO:26;
f) SEQ ID NO:27;
g) SEQ ID NO:28;
h) SEQ ID NO:29;
i) SEQ ID NO:30;
j) SEQ ID NO 31; and
k) SEQ ID NO: 32.

8. A composition comprising a first polynucleotide and a second polynucleotide according to claim 7, wherein the first polynucleotide contains an open reading frame encoding SEQ ID NO:29.

9. The isolated polynucleotide of claim 3, said fragment of SEQ ID NO:2 comprising at least one putative MNTF promoter sequence and at least one open reading frame.

10. The isolated polynucleotide of claim 9 wherein said putative MNTF promoter sequence is selected from the group consisting of:
a) residues 862-911 of SEQ ID NO:2; and
b) residues 2315-2364 of SEQ ID NO:2.

11. The isolated polynucleotide of claim 10 wherein said fragment of SEQ ID NO:2 includes a potential transcription start sequence comprising residues 2501-4359 of SEQ ID NO:2.

12. The isolated polynucleotide of claim 9 wherein the at least one open reading frame encodes a polypeptide selected from the group consisting of:
a) SEQ ID NO:13;
b) SEQ ID NO:14;
c) SEQ ID NO:15;
d) SEQ ID NO:16;
e) SEQ ID NO:17;
f) SEQ ID NO:18;
g) SEQ ID NO:19;
h) SEQ ID NO:20;
i) SEQ ID NO:21;
j) SEQ ID NO:22;
k) SEQ ID NO:23;
I) SEQ ID NO:24;
m) SEQ ID NO:25;
n) SEQ ID NO:26;
o) SEQ ID NO:27;
p) SEQ ID NO:28;
q) SEQ ID NO:29;
r) SEQ ID NO:30;
s) SEQ ID NO 31; and
t) SEQ ID NO: 32.

13. An isolated MNTF associated polypeptide encoded by an open reading frame of SEQ ID NO:1.

14. A fusion protein comprising an MNTF associated polypeptide encoded by an open reading frame of SEQ ID NO:1 linked to a heterologous protein.

15. An expression vector operably linked to the isolated polynucleotide according to claim 1, wherein at least one open reading frame is operably linked to a control sequence compatible with a desired host vector.

16. An isolated host cell transformed with the expression vector of claim 15.

17. A method for determining the presence of a MNTF associated polynucleotide in a medium comprising the steps of:
contacting the medium, which may contain an MNTF associated nucleic acid sequence, with a synthesized and isolated oligonucleotide which under preselected hybridization conditions hybridizes with said MNTF associated nucleic acid sequence, but does not hybridize with nucleic acid sequences other than said MNTF associated nucleic acid sequence, in said medium; and
detecting, under said preselected hybridization conditions, the presence of said MNTF associated nucleic acid sequence.

18. A method of comparing the relative abundance of MNTF associated expression products in different samples comprising:
obtaining a first sample and a second sample, wherein the first sample differs from the second sample;
detecting a MNTF related expression product for the first sample and the second sample; and comparing the relative abundance of the MNTF associated expression products of the first and second samples.

19. The method of claim 18, wherein MNTF RNA is the expression product.

20. The method of claim 18, wherein an MNTF associated polypeptide is the expression product.

21. The method of claim 18, wherein an MNTF associated polypeptide encoded by an open reading frame of SEQ ID NO:1 is the expression product.

22. The method of claim 18, wherein a polypeptide having at least SEQ ID NO. 29 is the expression product.

23. The method of claim 18, wherein the comparison includes a step comprising hybridization with a nucleic acid probe complementary to the RNA.

24. A panel for use in hybridization assays comprising two or more polynucleotides according to claim 1 stably associated with the surface of a solid support.

25. An isolated polynucleotide comprising a fragment of SEQ ID NO:1 or SEQ ID NO:2, provided that the nucleotide sequence of said fragment does not entirely correspond to SEQ ID NO:2 in U.S. Pat. No. 6,309,877.

26. An isolated polynucleotide comprising a fragment of SEQ ID NO:1 or SEQ ID NO:2, provided that the nucleotide sequence of said fragment does not entirely correspond to SEQ ID NO:5 in U.S. Pat. No. 6,309,877.


SEQUENCE LISTING (32 sequences)

SEQ ID NO: 1 - DNA, 1859 bp, Homo sapiens
cagctgctcagaggagtagctgggaccagggcccttcagtattctacctccagacccagcctaagcccacgctctctgagaaggccttgtatacactgaactccctcatgtatttcactgacaactgttttctgtgcccatgaagagttgcaagagcaccttgaaaacacaagcaaacatatcatatcaacttccaggcatctaattaattgtgcaccaaaatcgttacccttccaatcttgcaacagaagagcaaaagctgcttagaccttactcaaaggctccaaagcagaaacaagttttgcttcttgcagcaatgtgtaacagaggaaacagatgtcctggaagatcccatggtctgtttcaggtgagatggaaccaatgctgcacacgaagattcatctcaaatgaaatgcacaggaaaagccaattactttatgtgaataaaagaataaatccctaaagcagtggttctcaaccagagtgataccacccacaccccagagggcatttgggaattcttggggacattttggggtgacacactgaactgctggatgctatcagcatttagtaggtatgctcgatgtcttgcagaaggacatgatggtcctacacagtaaggaatggattacctacaatattaatagcagcctcccatacacacttttgacacccttccctaaaggattaatatgctccaaccttcctgtccccacagttcagtggctctccctaccctcaccatgatcggatgaaaaaaaataaggtttcacagcttaagagtgaaattctggaatccaactacaagctcataactgtagcatggaacctggtagtagcataataaataaatttttagtaagaggcttaagaaattttagcaaaaaaagcactccctttcttcctccctacatatctcatatgtttttcaacacaaaaaattctgtgattttagagaaacttcttacagtacttttaagttcaaaaccagatgctcattacagttcttttaaacaccaaactagtcatctcaaaaatatggctaactctctggactaaattccataggaaaaattattaatttcaaaatgcctaattttgatcaaatgcctgaaagagccaaaggcaatcatgtcctgcttctcactcagggcagagttccatgaggtcagaaaagctccaatgatatccggaggtctgtcagagattaaaatatcatcctaacaattcacaagctacttctaagtgttaccctaaattagtcactaatcgtttctcccccaacactatttcacaaattaaagtttacagaattgacaaaaaaccaaaccaaatgaaaacaaaccccaggctatttgcagggggggggaaagagataccccaaaagtcaaccctatttcacagtagttaaaagagtgatccaacagatattaccctccataaagtaccctaaaggcaggagcaatctttcccctacactatctaaattttcccaactcacccaaaagaatcttaagatgtaacttcatcactcaacttgccaattagcaaatagcggcccccacatcccttttggccaattttctagatgcctgacctagcagacagacctgttttcctcctgccaggccttttttgccctcccgctaggagcctgggaaatccccctccaaccttttgctggcctgtcaccagaagttcataaaatgaattcatccagattctctgaatcctccttttccccagtcatgtgccaacagccaggcaataatgccccaccgtgacaggctccctgttacaaaaagatgcagattcacgggaagaccaagcacttagcatggagcagaagaaaaaaa

SEQ ID NO: 2 - DNA, 4359 bp, Homo sapiens
accacagaatgcttttccttctccagaaaccatgtgaaggcccattacttctagcacacctatggcgggacacaaaggctttctgattcactccaagggagctggcaattctcccccattagctaacaataaaagaaaattaaatcacttgagaaccctacagtggcagcaaaatagagcccaagagatacctcctagcatgttcaaagacttattaaaagccacgttgggccaggcgtggtggcttatgcctataatcccaataccttgggaggttgaggtgggaggattgcttaaagcccggagtttgaggccagcctaggcaacatagtgagacctcatctctacaaaagattgtataaaatttagccaggtgtagtggtgcgcacctgagtcccagctactctggaagctgagatgggaagacggcttgagcccaagagtttgaggctacagtgaaccatgattgcaccatggcactccagcctgggcaacagagcaagaccctgtctcaaaaataaataaataacaaaaacaaaacacaatcaccttgtatcagtctcagttgatggcttgatgacaataaagaacagtttagactttctgaaagtgcatacatatatatacacatatgactaagaaggtgaagtgaagccctcaatggccagcccttttatagtagaacctagaaaacagcaacttctttcaacatggtggatggtgcatgttttgtaccccacatttagaaatccatactctgagacaaacagaactaccgaaagagaatgaccaggcctccagaatagcaccgaggcagccatcaggagttatagcaaaactttttaacgtggaaagggagtcactctgcttctataaagcagcccttatcagcggcattatgagtaaaagcagcccactaatgcaggccagactttaggacacagaaccagattccatttgggggctacagctagcacagctgtaaacctctctccacccatcaaattctgagaccattaggcctggcacggtggctcatgcctgtggtcccagcactttgggaagccaagctgggaggatcaactgagcccaggagtctgagactgcagtgagctatgatctcgccactgcactccagcctgggtgacagaataagaccctgtctcaaaagaaaaaaaaaaaaaatctgaggcgattgaaggcagacaactataagagagtgcccccataggaactccaccactctccatttagaatgtcctttgacttttgtgcacattatctcatgagaccctcactatgactctgagaggtaagcaggacaggcgtcttctacattttacagatgaataaattgtgactgaggttaaatgactcgcttgcccaaggtcacaaatcatgcagctaatcagctgcctaaccaggagatgagcctagcactctgactcgtaggccagcgcaatctccaggacaccatgcggctttgtgatgttttcagataagaggcggaagagggtactggttaagagcccaggttctggcaccagccaacctgcattccaatcccatctccaccacctactaaccacctgatcttgtgtgagttccttaacctccccaaacctgtttccttacctgcacaatgggggtgggagagaacagtcttactccatagagtcagagtgatgattaagtgagatgatgaacataaagtactcaaattcatgtactcaaaaaatgtgagtggcaggcacagtgcaggacccacaacagagatcaaggaacacagtcctgctctcacggagcttagactttaggcaaaccatacacagaaaaaagtaaacagataaaatacttacaaagtgtgttaagaatgattaaggtgcagggcgcagtggctcacgcctgtaatcctatcactttgggaggccgggtcaggcagatcacctgaggtcgggagtttgagaccagcctgataaacatggagaaatcccgtctctacaaaaaacaaaattagccccaagcttattgggcatggtggcgcatgccagtaatcccagctactcgggaggctgaggcaggagaattgcttgaacccgggaggcggagactgcagtgagcagaggtcgcaccactgcactccagcctgggcaacaagagcgaaactctgtctcaaaaaaaagaatgattaaggtaacaggtgagtgactggagagaataaccggaggggccaatttttaagagtggtctctaagccctaagggatcagaaagagaaattcgtgtgaagaacggagagaaaagcactctaggattagggaatggtatacgtgggggtcacagcacagggaagtcaggaacctagagaagacaaatgtgactacagcgcttcctctcccgggatttgcggagcaaggctggagctcgtgagggtgaccacaactattttaaaaacgacaaagataaggctcaagagaggggatatgtcttgcccaaggtcacagctgctcagaggagtagctgggaccagggcccttcagtattctacctccagacccagcctaagcccacgctctctgagaaggccttgtatacactgaactccctcatgtatttcactgacaactgttttctgtgcccatgaagagttgcaagagcaccttgaaaacacaagcaaacatatcatatcaacttccaggcatctaattaattgtgcaccaaaatcgttacccttccaatcttgcaacagaagagcaaaagctgcttagaccttactcaaaggctccaaagcagaaacaagttttgcttcttgcagcaatgtgtaacagaggaaacagatgtcctggaagatcccatggtctgtttcaggtgagatggaaccaatgctgcacacgaagattcatctcaaatgaaatgcacaggaaaagccaattactttatgtgaataaaagaataaatccctaaagcagtggttctcaaccagagtgataccacccacaccccagagggcatttgggaattcttggggacattttggggtgacacactgaactgctggatgctatcagcatttagtaggtatgctcgatgtcttgcagaaggacatgatggtcctacacagtaaggaatggattacctacaatattaatagcagcctcccatacacacttttgacacccttccctaaaggattaatatgctccaaccttcctgtccccacagttcagtggctctccctaccctcaccatgatcggatgaaaaaaaataaggtttcacagcttaagagtgaaattctggaatccaactacaagctcataactgtagcatggaacctggtagtagcataataaataaatttttagtaagaggcttaagaaattttagcaaaaaaagcactccctttcttcctccctacatatctcatatgtttttcaacacaaaaaattctgtgattttagagaaacttcttacagtacttttaagttcaaaaccagatgctcattacagttcttttaaacaccaaactagtcatctcaaaaatatggctaactctctggactaaattccataggaaaaattattaatttcaaaatgcctaattttgatcaaatgcctgaaagagccaaaggcaatcatgtcctgcttctcactcagggcagagttccatgaggtcagaaaagctccaatgatatccggaggtctgtcagagattaaaatatcatcctaacaattcacaagctacttctaagtgttaccctaaattagtcactaatcgtttctcccccaacactatttcacaaattaaagtttacagaattgacaaaaaaccaaaccaaatgaaaacaaaccccaggctatttgcagggggggggaaagagataccccaaaagtcaaccctatttcacagtagttaaaagagtgatccaacagatattaccctccataaagtaccctaaaggcaggagcaatctttcccctacactatctaaattttcccaactcacccaaaagaatcttaagatgtaacttcatcactcaacttgccaattagcaaatagcggcccccacatcccttttggccaattttctagatgcctgacctagcagacagacctgttttcctcctgccaggccttttttgccctcccgctaggagcctgggaaatccccctccaaccttttgctggcctgtcaccagaagttcataaaatgaattcatccagattctctgaatcctccttttccccagtcatgtgccaacagccaggcaataatgccccaccgtgacaggctccctgttacaaaaagatgcagattcacgggaagaccaagcacttagcatggagcagaagaaaaaaa

SEQ ID NO: 3 - DNA, 22 bp, Homo sapiens
tttcttcctccctacatctctc

SEQ ID NO: 4 - DNA, 21 bp, Homo sapiens
gagggtaatatctgttggatc

SEQ ID NO: 5 - DNA, 20 bp, Homo sapiens
ttggggacattttggggtga

SEQ ID NO: 6 - DNA, 20 bp, Homo sapiens
gctcgatgtcttgcagaagg

SEQ ID NO: 7 - DNA, 21 bp, Homo sapiens
agggtaacacttagaagtagc

SEQ ID NO: 8 - DNA, 19 bp, Homo sapiens
tcaccccaaaatgtcccca

SEQ ID NO: 9 - DNA, 27 bp, Artificial
  Vector primer
ctgttagcttggtaccgagctcggatc

SEQ ID NO: 10 - DNA, 27 bp, Homo sapiens
taggggaaagattgctcctgcctttag

SEQ ID NO: 11 - DNA, 27 bp, Homo sapiens
tattgcctggctgttggcacatgactg

SEQ ID NO: 12 - DNA, 27 bp, Homo sapiens
ctgctccatgctaagtgcttggtcttc

SEQ ID NO: 13 - protein, 24 aa, Homo sapiens
MSWKIPWSVSGEMEPMLHTKIHLK

SEQ ID NO: 14 - protein, 56 aa, Homo sapiens
MLDVLQKDMMVLHSKEWITYNINSSLPYTLLTPFPKGLICSNLPVPTVQWLSLPSP

SEQ ID NO: 15 - protein, 78 aa, Homo sapiens [sequence omitted]

SEQ ID NO: 16 - protein, 30 aa, Homo sapiens
MSCFSLRAEFHEVRKAPMISGGLSEIKISS

SEQ ID NO: 17 - protein, 21 aa, Homo sapiens
MPHRDRLPVTKRCRFTGRPST

SEQ ID NO: 18 - protein, 21 aa, Homo sapiens
MKSCKSTLKTQANISYQLPGI

SEQ ID NO: 19 - protein, 48 aa, Homo sapiens
MCNRGNRCPGRSHGLFQVRWNQCCTRRFISNEMHRKSQLLYVNKRINP

SEQ ID NO: 20 - protein, 6 aa, Homo sapiens
MSCRRT

SEQ ID NO: 21 - protein, 30 aa, Homo sapiens
MKKNKVSQLKSEILESNYKLITVAWNLVVA

SEQ ID NO: 22 - protein, 29 aa, Homo sapiens
MLITVLLNTKLVISKIWLTLWTKFHRKNY

SEQ ID NO: 23 - protein, 7 aa, Homo sapiens
MRSEKLQ

SEQ ID NO: 24 - protein, 25 aa, Homo sapiens
MKTNPRLFAGGGKEIPQKSTLFHSS

SEQ ID NO: 25 - protein, 38 aa, Homo sapiens
MPDLADRPVFLLPGLFCPPARSLGNPPPTFCWPVTRSS

SEQ ID NO: 26 - protein, 11 aa, Homo sapiens
MYFTDNCFLCP

SEQ ID NO: 27 - protein, 5 aa, Homo sapiens
MVCFR

SEQ ID NO: 28 - protein, 11 aa, Homo sapiens
MKCTGKANYFM

SEQ ID NO: 29 - protein, 21 aa, Homo sapiens
MLSAFSRYARCLAEGHDGPTQ

SEQ ID NO: 30 - protein, 20 aa, Homo sapiens
MLQPSCPHSSVALPTLTMIG

SEQ ID NO: 31 - protein, 25 aa, Homo sapiens
MPNFDQMPERAKGNHVLLLTQGRVP

SEQ ID NO: 32 - protein, 25 aa, Homo sapiens
MNSSRFSESSFSPVMCQQPGNNAPP